(19) United States
(12) Patent Application Publication
(10) Pub. No.: US 2025/0344661 A1
(43) Pub. Date: Nov. 13, 2025

(54) HYBRID PEPPER VARIETY 'SMK26'

(71) Applicant: BreedX LTD., Givat Brener (IL)

(72) Inventors: Binyamin NIR, Sderot (IL); David JOLLES, Eliav (IL)

(73) Assignee: BreedX LTD., Givat Brener (IL)

(21) Appl. No.: 19/201,224

(22) Filed: May 7, 2025

(30) Foreign Application Priority Data
May 10, 2024 (QZ) 2024/1093

(51) Int. Cl. A01H 6/82; A01H 5/08

(52) U.S. Cl. CPC A01H 6/822; A01H 5/08

(57) ABSTRACT

A new pepper hybrid designated as ‘SMK26’ is disclosed. ‘SMK26’ is a pepper variety exhibiting stability and uniformity.

### CROSS-REFERENCE TO RELATED APPLICATION

This application claims the benefit of Community Plant Variety Office (CPVO) Plant Variety Rights (PVR) Application No. 2024/1093, filed May 10, 2024, which is hereby incorporated by reference in its entirety.

### FIELD OF THE INVENTION

The present invention relates to the field of plant breeding. In particular, the present invention relates to new and distinctive pepper (Capsicum annuum L.) hybrid designated ‘SMK26’.

### BACKGROUND OF THE INVENTION

Peppers (Capsicum spp.) are members of the nightshade family (Solanaceae) and are important crop species in a variety of cultures. Though they originated in the tropical regions of the Americas, they are now grown and utilized globally as vegetables, spices, and medicines. Several species of the genus Capsicum are cultivated, with Capsicum annum L. being the most commonly grown pepper in the United States. Capsicum annum L. comprises many varieties that can be divided into two broad groups: hot peppers and sweet peppers. The fruits of hot peppers produce capsaicin, resulting in a pungent (hot) flavor, while the fruits of sweet peppers produce little to no capsaicin, resulting in a non-pungent (mild) flavor.

In temperate regions, pepper plants are grown as herbaceous annuals; in warmer regions where temperatures do not drop below freezing, they may be grown as perennials. Pepper plants may have a variety of growth habits, including erect, compact, or prostrate. They may exhibit determinate growth, with stems eventually terminating in flowers, or indeterminate growth, with stems continuously producing flowers without terminating. The leaves, which may take a variety of shapes from broadly ovate to ovate lanceolate, are shiny and glabrous and arranged alternately on the stem. The root system will form a deep taproot if uninjured, a secondary root system spreading laterally downward, and some adventitious roots at the soil surface. The flowers are white and develop either singly or in small clusters of two or three as the plant grows. The flowers may self-pollinate or be pollinated by insects, allowing for cross-pollination. The fruits may be a wide variety of shapes and colors at maturity, and are usually green prior to maturity, except for white and purple varieties which maintain the same color throughout growth and ripening. The seeds are kidney-shaped and develop on the interior surface of the hollow fruit flesh.

There are many different types of hot peppers and sweet peppers. One sweet pepper type is kapia pepper, which is a visually appealing pepper type originating from Turkey. Kapia pepper plants produce colorful fruit with a pointed horn shape. Mini kapia is a further developed pepper type that also produces colorful fruit with a pointed horn shape, but the produced fruit is shorter in length (8 to 15 cm).

There is a demand for seedless pepper varieties for the fresh market. In particular, there is a need for new hybrid seedless pepper varieties that are appealing to consumers and suitable for greenhouse cultivation. These varieties should be able to be cultivated in a variety of temperature conditions, such as the extreme temperature ranges found in passive greenhouses.

### SUMMARY OF THE INVENTION

In order to meet these needs, the present invention is directed to an improved hybrid seedless mini kapia pepper variety. This variety has very good fruit setting under normal and extreme temperatures, including both hot and cold conditions.

In one aspect, the present disclosure is directed to a hybrid pepper, Capsicum annuum L., seed designated as ‘SMK26’ having NCIMB Accession Number X1. In an embodiment of this aspect, the present disclosure is directed to a Capsicum annuum L. pepper plant and parts isolated therefrom produced by growing ‘SMK26’ pepper seed. In another embodiment of this aspect, the present disclosure is directed to a Capsicum annuum L. plant and parts isolated therefrom having all the physiological and morphological characteristics of a Capsicum annuum L. plant produced by growing ‘SMK26’ pepper seed having NCIMB Accession Number X1. In still another embodiment of this aspect, the present disclosure is directed to a method of making pepper seed, the method comprising crossing a ‘SMK26’ pepper plant with another pepper plant and harvesting seed therefrom.

Pepper plant parts include pepper leaves, shoots, stems, roots, cuttings, flowers, ovules, pollen, seeds, pepper fruits, parts of pepper fruits, cells, cotyledons, hypocotyls, meristems, and the like. In one embodiment, the present disclosure is further directed to a leaf, an ovule, a pollen grain, a fruit, or a cell isolated from ‘SMK26’ pepper plants. In a further embodiment of this aspect, the present disclosure is directed to a fruit isolated from ‘SMK26’ pepper plants. In certain embodiments, the present disclosure is further directed to pollen or ovules isolated from ‘SMK26’ pepper plants. In another embodiment, the present disclosure is further directed to protoplasts produced from ‘SMK26’ pepper plants. In another embodiment, the present disclosure is further directed to tissue or cell culture of ‘SMK26’ pepper plants, and to pepper plants regenerated from the tissue or cell culture, wherein the plants have all of the morphological and physiological characteristics of ‘SMK26’ pepper plants. In certain embodiments, tissue culture of ‘SMK26’ pepper plants is produced from a plant part selected from root, root tip, meristematic cell, stem, hypocotyl, petiole, cotyledon, leaf, flower, anther, pollen, pistil, and fruit.

In a further aspect, the present disclosure is directed to methods of producing a doubled haploid plant, including: (a) producing a haploid genome from the seed of any of the preceding embodiments or from a plant produced by growing the seed of any of the preceding embodiments; (b) doubling the haploid genome to produce a doubled haploid genome; and (c) producing a plant comprising the doubled haploid genome, thereby producing a doubled haploid plant. Some embodiments of this aspect relate to a doubled haploid plant produced from these methods. Additional embodiments of this aspect relate to a plant part from the doubled haploid plant, wherein said part is a leaf, an ovule, a pollen grain, a fruit, a cell, or a seed.

In still another aspect, the present disclosure is directed to methods of producing a seed of a ‘SMK26’-derived pepper plant, including the steps of: (a) producing a haploid genome from a hybrid pepper designated as ‘SMK26’; (b) doubling the haploid genome to produce a doubled haploid genome; (c) producing a plant comprising the doubled haploid genome, thereby producing a doubled haploid ‘SMK26’ pepper; (d) crossing the doubled haploid ‘SMK26’ pepper with itself or a second pepper plant; and (e) whereby seed of a ‘SMK26’-derived pepper plant forms. In another embodiment of this aspect, the method further includes the steps of: (f) crossing a plant grown from ‘SMK26’-derived pepper seed with itself or a second pepper plant to yield additional ‘SMK26’-derived pepper seed; (g) growing the additional ‘SMK26’-derived pepper seed of step (c) to yield additional ‘SMK26’-derived pepper plants; and (h) repeating steps (f) and (g) for an additional 3-10 generations to generate further ‘SMK26’-derived pepper plants. Some embodiments of this aspect relate to a pepper plant or seed produced from the methods of any of the preceding embodiments, wherein the pepper plant or seed is a doubled haploid or a polyploid of any ploidy level.

In yet another aspect, the present disclosure is directed to a method of vegetatively propagating a plant produced by growing the seed of hybrid pepper designated as ‘SMK26’, the method including the steps of: (a) collecting tissue capable of being propagated from a plant of hybrid pepper ‘SMK26’, representative sample of seed having been deposited under NCIMB Accession Number X1; (b) cultivating the tissue to obtain proliferated shoots; and (c) rooting the proliferated shoots to obtain rooted plantlets. In a further embodiment of this aspect, the method further includes step (d) growing plants from the rooted plantlets. In another embodiment, the present disclosure is further directed to pepper plants, plant parts and seeds produced by the pepper plants where the pepper plants are produced by any of the preceding methods of the disclosure.

In another embodiment, the present disclosure is directed to single gene converted plants of hybrid pepper ‘SMK26’. The single transferred gene may preferably be a dominant or recessive allele. Preferably, the single transferred gene will confer such trait as sex determination, herbicide resistance, insect resistance, resistance for bacterial, fungal, or viral disease, improved harvest characteristics, enhanced nutritional quality, or improved agronomic quality. The single gene may be a naturally occurring pepper gene or a transgene introduced through genetic engineering techniques.

In addition to the exemplary aspects and embodiments described above, further aspects and embodiments will become apparent by reference by study of the following descriptions.

### BRIEF DESCRIPTION OF THE DRAWINGS

The patent or application file contains at least one drawing executed in color. Copies of this patent or patent application publication with color drawings will be provided by the office upon request and payment of the necessary fee.

FIGS. 1A-1B show the upper and lower surfaces of leaves of hybrid pepper ‘SMK26’. FIG. 1A shows the upper surface of a leaf of hybrid pepper ‘SMK26’. FIG. 1B shows the lower surface of a leaf of hybrid pepper ‘SMK26’.

FIGS. 2A-2B show top and bottom views of flowers of hybrid pepper ‘SMK26’. FIG. 2A shows a top view of a flower of hybrid pepper ‘SMK26’. FIG. 2B shows a bottom view of a flower of hybrid pepper ‘SMK26’.

FIGS. 3A-3B show fruit of hybrid pepper ‘SMK26’. FIG. 3A shows an immature fruit of hybrid pepper ‘SMK26’. FIG. 3B shows a mature fruit of hybrid pepper ‘SMK26’.

FIGS. 4A-4B show horizontal and vertical cross-sections of mature fruit of hybrid pepper ‘SMK26’. FIG. 4A shows a horizontal cross-section of a mature fruit of hybrid pepper ‘SMK26’.FIG. 4B shows a vertical cross-section of mature fruit of hybrid pepper ‘SMK26’.

FIG. 5 shows whole plants of hybrid pepper ‘SMK26’.

### DETAILED DESCRIPTION OF THE INVENTION

There are numerous steps in the development of novel, desirable pepper germplasm. Plant breeding begins with the analysis of problems and weaknesses of current pepper germplasms, the establishment of program goals, and the definition of specific breeding objectives. The next step is selection of germplasm that possess the traits to meet the program goals. The goal is to combine in a single variety or hybrid an improved combination of desirable traits from the parental germplasm. These important traits may include higher yield, field performance, and resistance to diseases and insects. These important traits may also include fruit and agronomic quality such as fruit shape, fruit appearance, fruit size, and fruit capsaicin levels.

Choice of breeding or selection methods can depend on the mode of plant reproduction, the heritability of the trait(s) being improved, and the type of variety used commercially (e.g., F₁ hybrid variety, pureline variety, etc.). For highly heritable traits, a choice of superior individual plants evaluated at a single location will be effective, whereas for traits with low heritability, selection should be based on mean values obtained from replicated evaluations of families of related plants. Popular selection methods commonly include pedigree selection, modified pedigree selection, mass selection, and recurrent selection.

The complexity of inheritance influences choice of the breeding method. Backcross breeding is used to transfer one or a few favorable genes for a highly heritable trait into a desirable variety. This approach has been used extensively for breeding disease-resistant varieties. Various recurrent selection techniques are used to improve quantitatively inherited traits controlled by numerous genes. The use of recurrent selection in self-pollinating crops depends on the ease of pollination, the frequency of successful hybrids from each pollination, and the number of hybrid offspring from each successful cross.

Each breeding program may include a periodic, objective evaluation of the efficiency of the breeding procedure. Evaluation criteria vary depending on the goal and objectives, and can include gain from selection per year based on comparisons to an appropriate standard, the overall value of the advanced breeding lines, and the number of successful varieties produced per unit of input (e.g., per year, per dollar expended, etc.).

Promising advanced breeding lines may be thoroughly tested and compared to appropriate standards in environments representative of the commercial target area(s) for at least three years. The best lines can then be candidates for new commercial varieties. Those still deficient in a few traits may be used as parents to produce new populations for further selection. These processes, which lead to the final step of marketing and distribution, may take from ten to twenty years from the time the first cross or selection is made.

One goal of pepper plant breeding is to develop new, unique, and genetically superior pepper varieties. A breeder can initially select and cross two or more parental lines, followed by repeated selfing and selection, producing many new genetic combinations. Moreover, a breeder can generate multiple different genetic combinations by crossing, selfing, and mutations. A plant breeder can then select which germplasms to advance to the next generation. These germplasms may then be grown under different geographical, climatic, and soil conditions, and further selections can be made during, and at the end of, the growing season.

The development of commercial pepper varieties thus requires the development of parental pepper varieties, the crossing of these varieties, and the evaluation of the crosses. Pedigree breeding and recurrent selection breeding methods may be used to develop varieties from breeding populations. Breeding programs can be used to combine desirable traits from two or more varieties or various broad-based sources into breeding pools from which new varieties are developed by selfing and selection of desired phenotypes. The new varieties are crossed with other varieties and the hybrids from these crosses are evaluated to determine which have commercial potential.

Pedigree breeding is generally used for the improvement of self-pollinating crops or inbred lines of cross-pollinating crops. Two parents which possess favorable, complementary traits are crossed to produce an F₁. An F₂ population is produced by selfing one or several F₁s or by intercrossing two F₁s (sib mating). Selection of the best individuals is usually begun in the F₂ population. Then, beginning in the F₃, the best individuals in the best families are selected. Replicated testing of families, or hybrid combinations involving individuals of these families, often follows in the F₄ generation to improve the effectiveness of selection for traits with low heritability. At an advanced stage of inbreeding (i.e., F₆ and F₇), the best lines or mixtures of phenotypically similar lines are tested for potential release as new varieties.

Mass and recurrent selections can be used to improve populations of either self- or cross-pollinating crops. A genetically variable population of heterozygous individuals is either identified or created by intercrossing several different parents. The best plants are selected based on individual superiority, outstanding progeny, or excellent combining ability. The selected plants are intercrossed to produce a new population in which further cycles of selection are continued.

Backcross breeding may be used to transfer genes for a simply inherited, highly heritable trait into a desirable homozygous cultivar or line that is the recurrent parent. The source of the trait to be transferred is called the donor parent. The resulting plant is expected to have the attributes of the recurrent parent (e.g., cultivar) and the desirable trait transferred from the donor parent. After the initial cross, individuals possessing the phenotype of the donor parent are selected and repeatedly crossed (backcrossed) to the recurrent parent. The resulting plant is expected to have the attributes of the recurrent parent (e.g., cultivar) and the desirable trait transferred from the donor parent.

The single-seed descent procedure in the strict sense refers to planting a segregating population, harvesting a sample of one seed per plant, and using the one-seed sample to plant the next generation. When the population has been advanced from the F₂ to the desired level of inbreeding, the plants from which lines are derived will each trace to different F₂ individuals. The number of plants in a population declines each generation due to failure of some seeds to germinate or some plants to produce at least one seed. As a result, not all of the F₂ plants originally sampled in the population will be represented by a progeny when generation advance is completed.

In addition to phenotypic observations, the genotype of a plant can also be examined. There are many laboratory-based techniques known in the art that are available for the analysis, comparison and characterization of plant genotype. Such techniques include, without limitation, DNA- or RNA-sequencing, CAPS Markers, ELISA, Western blot, microarrays, Single Nucleotide Polymorphisms (SNPs), Isozyme Electrophoresis, Restriction Fragment Length Polymorphisms (RFLPs), Randomly Amplified Polymorphic DNAs (RAPDs), Arbitrarily Primed Polymerase Chain Reaction (AP-PCR), Differential Display Polymerase Chain Reaction (DD-PCR), Quantitative Real-Time Polymerase Chain Reaction (qRT-PCR), DNA Amplification Fingerprinting (DAF), Sequence Characterized Amplified Regions (SCARs), Amplified Fragment Length Polymorphisms (AFLPs), and Simple Sequence Repeats (SSRs, which are also referred to as Microsatellites).

Molecular markers can also be used during the breeding process for the selection of qualitative traits. For example, markers closely linked to alleles or markers containing sequences within the actual alleles of interest can be used to select plants that contain the alleles of interest during a backcrossing breeding program. The markers can also be used to select toward the genome of the recurrent parent and against the markers of the donor parent. This procedure attempts to minimize the amount of genome from the donor parent that remains in the selected plants. It can also be used to reduce the number of crosses back to the recurrent parent needed in a backcrossing program. The use of molecular markers in the selection process is often called genetic marker enhanced selection or marker-assisted selection. Molecular markers may also be used to identify and exclude certain sources of germplasm as parental varieties or ancestors of a plant by providing a means of tracking genetic profiles through crosses.

Mutation breeding may also be used to introduce new traits into pepper varieties. Mutations that occur spontaneously or are artificially induced can be useful sources of variability for a plant breeder. The goal of artificial mutagenesis is to increase the rate of mutation for a desired characteristic. Mutation rates can be increased by many different means including temperature, long-term seed storage, tissue culture conditions, radiation (such as X-rays, Gamma rays, neutrons, Beta radiation, or ultraviolet radiation), chemical mutagens (such as base analogs like 5-bromo-uracil), antibiotics, alkylating agents (such as sulfur mustards, nitrogen mustards, epoxides, ethyleneamines, sulfates, sulfonates, sulfones, or lactones), azide, hydroxylamine, nitrous acid, or acridines. Once a desired trait is observed through mutagenesis the trait may then be incorporated into existing germplasm by traditional breeding techniques. Details of mutation breeding can be found in Principles of Cultivar Development by Fehr, Macmillan Publishing Company (1993).

The production of double haploids can also be used for the development of homozygous varieties in a breeding program. Double haploids are produced by the doubling of a set of chromosomes from a heterozygous plant to produce a completely homozygous individual. For example, see Wan, et al., Theor. Appl. Genet., 77:889-892 (1989).

Additional non-limiting examples of breeding methods that may be used include, without limitation, those found in Principles of Plant Breeding, John Wiley and Son, pp. 115-161 (1960); Allard (1960); Simmonds (1979); Sneep, et al. (1979); Fehr (1987); and Genetics, Genomics and Breeding of Peppers and Eggplants, Kang & Kole, CRC Press (2013).

### Definitions

In the description and tables that follow, a number of terms are used. In order to provide a clear and consistent understanding of the specification and claims, including the scope to be given such terms, the following definitions are provided:

Backcrossing. Backcrossing is a process in which a breeder repeatedly crosses hybrid progeny back to one of the parents, for example, a first generation hybrid F₁ with one of the parental genotype of the F₁ hybrid.

Essentially all the physiological and morphological characteristics. A plant having essentially all the physiological and morphological characteristics means a plant having the physiological and morphological characteristics of the recurrent parent, except for the characteristics derived from the converted gene.

Internode. An “internode” refers to the stem segment between nodes.

Pepper fruit. As used herein, a “pepper fruit” is a fruit produced by a Capsicum annuum L. plant and is commonly referred to as a pepper. The color of a pepper fruit can be green, red, yellow, orange and, more rarely, white, black, and brown, depending on when they are harvested and the specific cultivar.

Propagate. To “propagate” a plant means to reproduce the plant by means including, but not limited to, seeds, cuttings, divisions, tissue culture, embryo culture or other in vitro method.

Regeneration. As used herein, “regeneration” refers to the development of a plant from tissue culture.

Single gene converted. As used herein, “single gene converted” or “conversion plant” refers to plants which are developed by a plant breeding technique called backcrossing wherein essentially all of the desired morphological and physiological characteristics of an inbred are recovered in addition to the single gene transferred into the inbred via the backcrossing technique or via genetic engineering.

Sweet pepper. As used herein, “sweet pepper” refers to a pepper plant, or fruits thereof, that lack sufficient capsaicin to produce a pungent flavor.

### Overview of the Hybrid Pepper Variety ‘SMK26’

Hybrid pepper ‘SMK26’ is a seedless mini kapia sweet pepper that produces mature fruit having a triangular shape and a red color. This variety is suitable for growing in greenhouses. Additionally, hybrid pepper ‘SMK26’ is moderately resistant to cracking and stip, and highly resistant to heat and cold. In particular, hybrid pepper ‘SMK26’ has very good fruit setting under both normal and extreme temperatures, including both hot and cold conditions such as those typical of passive greenhouses in Almeria during the summer and winter. ‘SMK26’ is especially suited for growth under hot conditions. Hybrid pepper ‘SMK26’ is the result of numerous generations of plant selections for its two parent lines, and was chosen for its very good fruit setting in hot and cold growing conditions, as well as its high resistance to TMV3 (PMMV) and moderate resistance to TSWV and powdery mildew.

Hybrid pepper ‘SMK26’ originated from a controlled cross between varieties ‘PF123’ (unpatented) and ‘PF511’ (unpatented). The breeding method used to develop ‘SMK26’ was single plant selection. The female parent ‘PF123’ was chosen from the fifth filial generation. The male parent was initially identified in the fourth filial generation, and single plant selection was then continued until the sixth filial generation, at which time ‘PF511’ was selected. ‘SMK26’ is a uniform F1 hybrid. The breeder's reference names for ‘SMK26’ are “CAG242-15X123-b-F1” and “CSCAG2311-40X33-F1”.

The variety has shown uniformity and stability for the traits, within the limits of environmental influence for the traits. Hybrid pepper ‘SMK26’ has been increased with continued observation for uniformity. No variant traits have been observed or are expected in ‘SMK26’.

### Variety Description of Hybrid Pepper ‘SMK26’

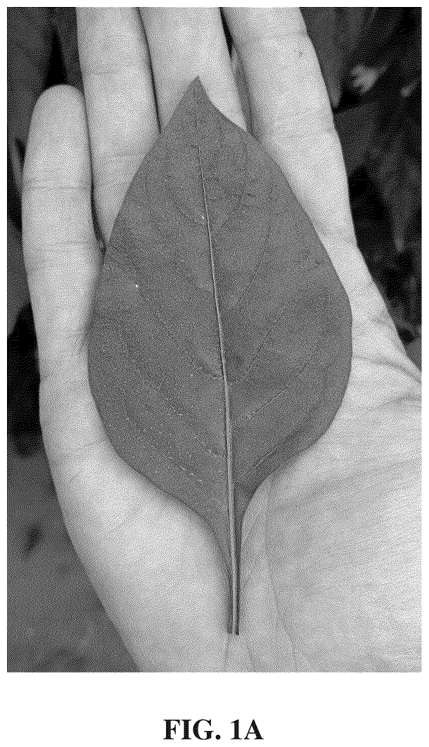

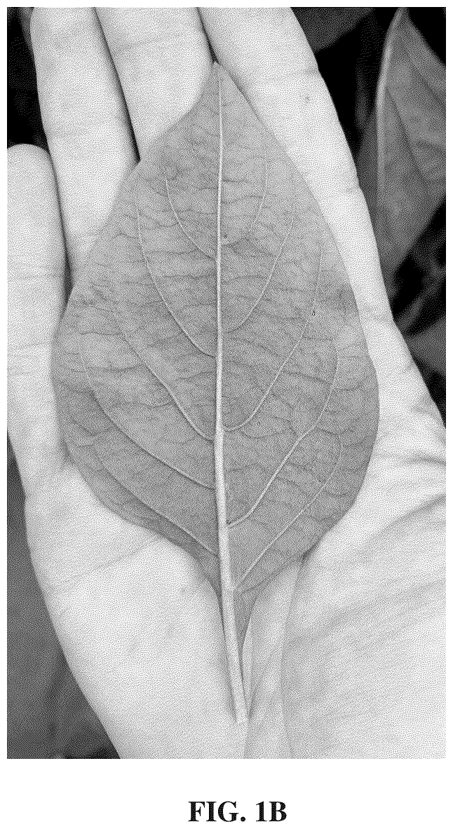

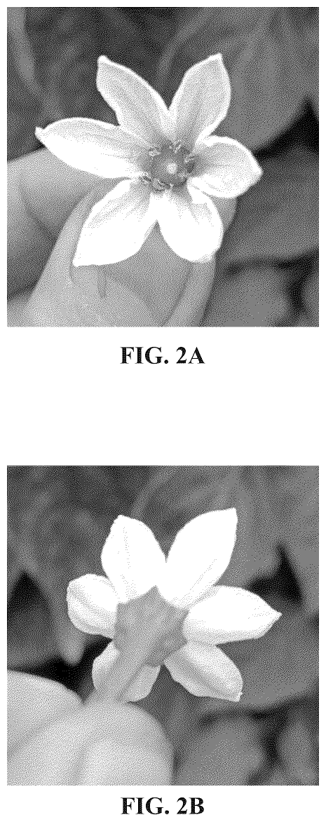

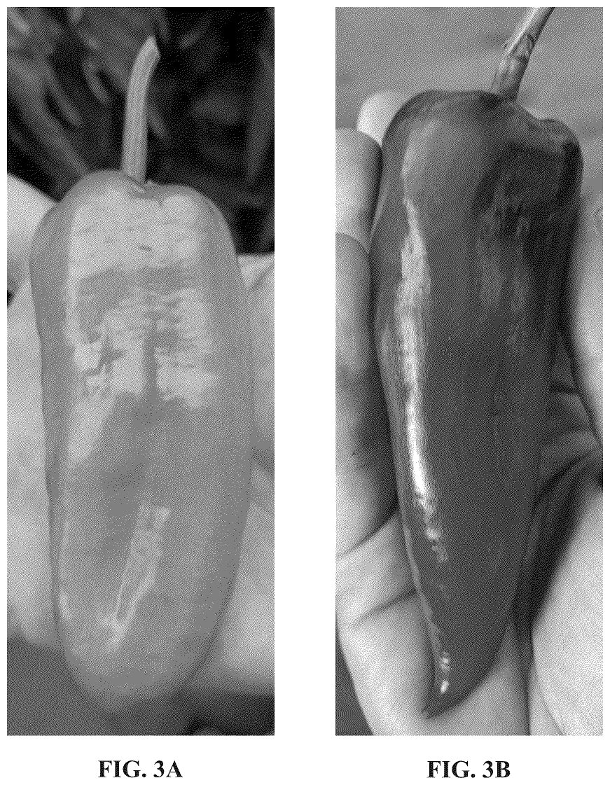

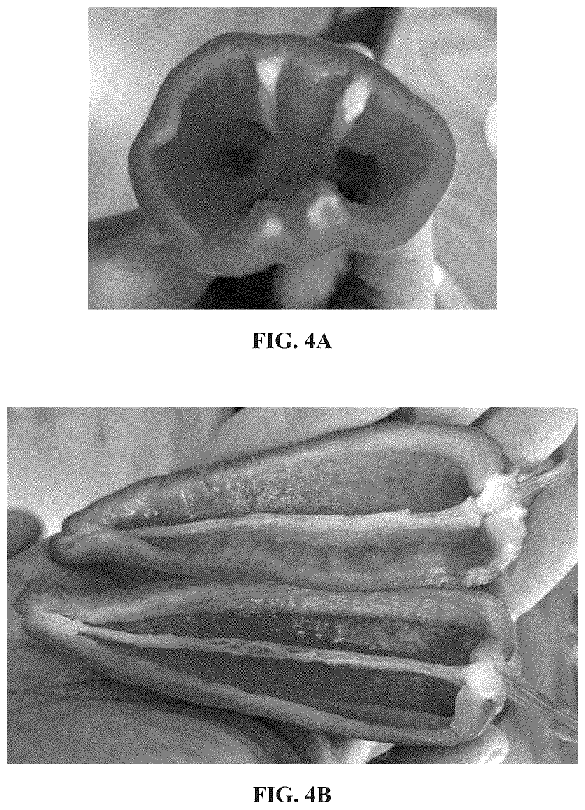

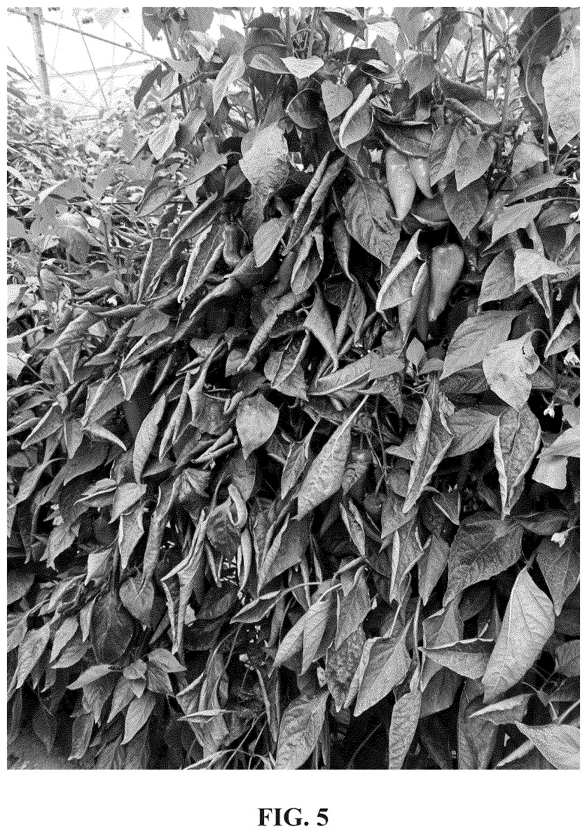

The variety description below provides a summary of hybrid pepper variety ‘SMK26’ characteristics. Those of skill in the art will recognize that these are typical values that may vary due to environment, and that other values that are substantially equivalent are within the scope of the invention. The terminology and descriptors are in line with the descriptors of the “UPOV Guidelines for the Conduct of Tests for Distinctness, Uniformity, and Stability”, or the “Test Guidelines” for Theobroma cacao L. The “Test Guidelines” indicate reference varieties for the descriptors or characteristics that are included in the list. The terminology and descriptors used in these tables are in line with the official terminology as of the filing date, and are thus clear for a person skilled in the art. FIGS. 1A-1B show upper and lower surfaces of leaves of hybrid pepper ‘SMK26’. FIGS. 2A-2B show top and bottom views of flowers of hybrid pepper ‘SMK26’. FIGS. 3A-3B show immature and mature fruit of hybrid pepper ‘SMK26’. FIGS. 4A-4B show horizontal and vertical cross-sections of fruit of hybrid pepper ‘SMK26’. FIG. 5 shows whole plants of hybrid pepper ‘SMK26’.

The following detailed descriptions set forth the distinctive characteristics of hybrid pepper variety ‘SMK26’. The data which define these characteristics is based on observations taken in Israel on plants 10 to 11 weeks after transplanting. This description is in accordance with UPOV terminology. Color designations, color descriptions, and other phenotypical descriptions may deviate from the stated values and descriptions depending upon variation in environmental, seasonal, climatic, and cultural conditions. ‘SMK26’ has not been observed under all possible environmental conditions. The indicated values represent averages calculated from measurements of several plants. Descriptive terminology follows the Plant Identification Terminology, An Illustrated Glossary, 2^{nd} edition by James G. Harris and Melinda Woolf Harris, unless where otherwise defined

Type: Mini kapia; seedless

### Seedling:

Anthocyanin coloration of hypocotyl: Present

### Three-Week-Old Plant:

Plant height: 15 cm
Stem:
   Length: 5 cm
   Pubescence: Very weak
Leaf:
   Length: 4.5 cm
   Width: 3.5 cm
   Color: Medium green
   Pubescence: Very weak
   Margin undulation: Medium
   Profile in cross section: Strongly concave
   Petiole length: 3.5 cm

### Plant:

Habit (without pruning or training): Upright
Attitude: Erect
Growth: Indeterminate
Height: 170 cm
Stem:
   Length: 11 to 15 cm
   Pubescence: Absent or very weak to very weak to weak
   Intensity of anthocyanin coloration of nodes: Weak to medium
   Strength: Strong
Shortened internode: Absent
Length of internode (measured on the upper part after the first branching of the main axis on primary side shoots): Short to medium
Basal branches: None to few
Branch flexibility: Rigid

### Leaf:

Length: 16 to 20 cm
Width: 8 to 9 cm
Length to width ratio: Medium
Shape: Ovate
Color: Medium green to dark green
Intensity of anthocyanin coloration of upper side (of recently fully developed leaves): Absent or very weak to weak
Distribution of anthocyanin coloration of lower side (of recently fully developed leaves): Absent
Leaf blade variegation: Absent
Pubescence: Weak
Margin undulation: Weak
Blistering: Very weak
Glossiness: Very weak to weak
Profile in cross section: Moderately concave
Petiole length: 8 to 10 cm

### Flower:

Number of flowers per leaf axil: 1 to 3
Number of calyx lobes: 5
Number of petals: 5 to 6
Flower diameter: 25 mm
Corolla color: White
Corolla throat markings: White
Anthocyanin coloration of anther: Absent
Anthocyanin coloration of filament: Absent
Style length: Same as stamen to longer than stamen
Self-incompatibility: Absent
Time of beginning of flowering (when 50% of plants have first open flower of the second flowering node): Very early to early
Predominant attitude of pedicel: Semi-drooping
Male sterility: Totally present

### Fruit:

Immature fruit color: Light green
Anthocyanin coloration of immature fruit: Absent or weak
Mature (ripe) fruit color: Red
Intensity of mature fruit follow: Medium to dark
Pungency: Sweet (capsaicin absent from placenta)
Flavor: Moderate pepper flavor
Glossiness: Weak to medium
Surface texture: Smooth or weakly wrinkled
Attitude: Drooping
Calyx:
   Shape: Saucer-shaped
   Aspect: Semi-enveloping
   Anthocyanin coloration: Absent
Length: 8 to 13 cm
Diameter at midpoint: 2.5 to 3.5 cm
Fruit length/diameter ratio: Very low to low or low
Depth of stalk cavity: Absent or very shallow to very shallow to shallow
Flesh thickness at midpoint: 2 to 3 mm
Average number of fruit per plant: 50 to 60
Fruit set: Scattered
Average fruit weight: 23 to 30 grams
Base shape: Rounded
Apex shape: Moderately acute
Shape in longitudinal section: Triangular
Curvature: Absent
Twisting: Absent or weak
Shape in cross section (at level of placenta): Angular
Sinuation of pericarp at basal part: Absent or very weak
Sinuation of pericarp excluding basal part: Medium
Depth of interloculary grooves (at middle third of the fruit): Very shallow to shallow
Average number of locules: Predominantly two
Pedicel:
   Length: 3 to 4 cm
   Width: 0.5 cm
   Shape: Curved

### Growth Conditions:

Type of culture: Greenhouse
Growing system: Suitable for hydroponic growing system
Principle use: Fresh market
Machine harvest: Not suitable
Time of maturity: Very early to early

### Resistance to Diseases and Pests:

Tobacco mosaic virus (TMV) Pathotype 3 (TMV3; PMMV): Highly resistant
Tobacco mosaic virus (TMV) Pathotype 0 (TMVO): Resistant
Tomato spotted wilt virus (TSWV) Pathotype 0 (TSWV 0): Moderately resistant
Powdery mildew (Leveillula taurica (anamorph: Oidiopsis sicula)): Moderately resistant
Pepper mild mottle virus (PMMoV) Pathotype 1-2 (PMMoV 1-2): Resistant
Pepper mild mottle virus (PMMoV) Pathotype 1-2-3 (PMMoV 1-2-3): Resistant

### Resistance to Abiotic Stress:

Cracking: Moderately resistant
Stip: Moderately resistant
Heat: Highly resistant
Cold: Highly resistant

### Comparison to Most Similar Variety

Table 1 below compares some of the characteristics of hybrid pepper variety ‘SMK26’ with similar variety, ‘SMK3’ (CPVO Plant Variety Right Grant Certificate No. 57493). Column 1 lists the characteristics, column 2 shows the characteristics for hybrid pepper variety ‘SMK26’, and column 3 shows the characteristics for most similar pepper variety ‘SMK3’.

TABLE 1

| Characteristic | ‘SMK26’ | ‘SMK3’ |
|---|---|---|
| Fruit length | 8 to 15 cm | 8 to 15 cm |
| Ability to set fruit at extreme | Excellent fruit setting | Good fruit setting |
| temperatures: cold conditions | | |
| Ability to set fruit at extreme | Excellent fruit setting | Very low ability |
| temperatures: hot conditions | | to set fruit |

### Further Embodiments

This disclosure also is directed to methods for producing a pepper plant by crossing a first parent pepper plant with a second parent pepper plant wherein either the first or second parent pepper plant is a hybrid pepper plant of ‘SMK26’. Further, both first and second parent pepper plants can come from the hybrid pepper ‘SMK26’. All plants produced using hybrid pepper ‘SMK26’ as a parent are within the scope of this disclosure, including plants derived from hybrid pepper ‘SMK26’. Plants derived from hybrid pepper ‘SMK26’ may be used, in certain embodiments, for the development of new pepper varieties. By selecting plants having one or more desirable traits, a plant derived from hybrid pepper ‘SMK26’ is obtained which possesses some of the desirable traits of the hybrid as well as potentially other selected traits.

The development of new varieties using one or more starting varieties is well known in the art. In accordance with this disclosure, novel varieties may be created by crossing hybrid pepper ‘SMK26’ followed by multiple generations of breeding according to such well known methods as well as using double haploid (DH) technology to have homozygous lines from the novel varieties created by crossing hybrid pepper SMK26 with any other line and/or hybrid pepper. New varieties may be created by crossing with any second plant. In selecting such a second plant to cross for the purpose of developing novel lines, it may be desired to choose those plants which either themselves exhibit one or more selected desirable characteristics or which exhibit the desired characteristic(s) when in hybrid combination. Once initial crosses have been made, inbreeding and selection take place to produce new varieties. For development of a uniform line, often five or more generations of selfing and selection are involved.

It is preferred to breed for a combination of desirable plant characteristics and resistances to create a single variety or hybrid containing an improved combination of desirable traits from the parental germplasm. The development of commercial pepper hybrids relates to the development of pepper parental lines, the crossing of these lines, and the evaluation of the crosses. Hybrid varieties offer multiple advantages, including a combination of desirable dominant and recessive traits from a set of inbred parents. Pedigree breeding, double haploid (DH), and recurrent selection breeding methods are used to develop cultivars from breeding populations. Breeding programs combine desirable traits from two or more varieties or various broad-based sources into breeding pools from which lines are developed by DH, selfing, and/or selection of desired phenotypes. The new lines are crossed with other lines and the hybrids from these crosses are evaluated to determine which have the desirable characteristics.

### Gene Conversions

When the terms “pepper plant”, “hybrid”, “cultivar”, or “pepper line” are used in the context of the present invention, this also includes any gene conversions of that variety. The term “gene converted plant” as used herein refers to those pepper plants which are developed by backcrossing, genetic engineering, DH, or mutation, where essentially all of the desired morphological and physiological characteristics of a variety are recovered in addition to the one or more genes transferred into the variety via the backcrossing technique, genetic engineering, or mutation. Backcrossing methods can be used with the present invention to improve or introduce a characteristic into the variety. The term “backcrossing” as used herein refers to the repeated crossing of a hybrid progeny back to the recurrent parent, i.e., backcrossing 1, 2, 3, 4, 5, 6, 7, 8, 9, or more times to the recurrent parent. The parental pepper plant which contributes the gene for the desired characteristic is termed the “nonrecurrent” or “donor parent.” This terminology refers to the fact that the nonrecurrent parent is used one time in the backcross protocol and therefore does not recur. The parental pepper plant to which the gene or genes from the nonrecurrent parent are transferred is known as the recurrent parent, as it is used for several rounds in the backcrossing protocol (Poehlman & Sleper (1994) and Fehr (1993)). In a typical backcross protocol, the original variety of interest (recurrent parent) is crossed to a second variety (nonrecurrent parent) that carries the gene of interest to be transferred. The resulting progeny from this cross are then crossed again to the recurrent parent and the process is repeated until a pepper plant is obtained where essentially all of the desired morphological and physiological characteristics of the recurrent parent are recovered in the converted plant, in addition to the transferred gene from the nonrecurrent parent.

The selection of a suitable recurrent parent is an important step for a successful backcrossing procedure. The goal of a backcross protocol is to alter or substitute a trait or characteristic in the original line. To accomplish this, a gene of the recurrent variety is modified or substituted with the desired gene from the nonrecurrent parent, while retaining essentially all of the rest of the desired genetic, and therefore the desired physiological and morphological, constitution of the original line. The choice of the particular nonrecurrent parent will depend on the purpose of the backcross. One of the major purposes is to add some commercially desirable, agronomically important trait to the plant. The exact backcrossing protocol will depend on the characteristic or trait being altered, which will determine an appropriate testing protocol. Although backcrossing methods are simplified when the characteristic being transferred is a dominant allele, a recessive allele may also be transferred. In this instance it may be necessary to introduce a test of the progeny to determine if the desired characteristic has been successfully transferred.

Many gene traits have been identified that are not regularly selected in the development of a new line but that can be improved by backcrossing techniques. Examples of these traits include, but are not limited to, male sterility, modified fatty acid metabolism, modified carbohydrate metabolism, herbicide resistance, resistance for bacterial, fungal, or viral disease, insect resistance, enhanced nutritional quality, industrial usage, yield stability, and yield enhancement. These genes are generally inherited through the nucleus. Several of these gene traits are described in U.S. Pat. Nos. 5,777,196, 5,948,957, and 5,969,212.

### Tissue Culture

As used herein, the term “tissue culture” indicates a composition containing isolated cells of the same or a different type or a collection of such cells organized into parts of a plant. Exemplary types of tissue cultures are protoplasts, calli, meristematic cells, and plant cells that can generate tissue culture that are intact in plants or parts of plants, such as leaves, pollen, embryos, roots, root tips, anthers, pistils, flowers, seeds, petioles, suckers, and the like. Means for preparing and maintaining plant tissue culture are well known in the art. By way of example, a tissue culture containing organs has been used to produce regenerated plants. U.S. Pat. Nos. 5,959,185, 5,973,234, and 5,977,445 describe certain techniques, which may be used.

Tissue culture of pepper can be used for the in vitro regeneration of pepper plants. Tissue cultures of various tissues of pepper and regeneration of plants therefrom are well-known and published. By way of example, tissue cultures, some comprising organs to be used to produce regenerated plants, as well as regeneration by somatic embryogenesis, have been described in Gammoudi, et al., In Vitro Cellular & Developmental Biology—Plant, 54, 145-153 (2018); Kothari, et al., Biotechnology Advances, 28:1, 34-48 (2010); Ochoa-Alejo and Ramirez-Malagon, In Vitro Cellular & Developmental Biology—Plant, 37, 701-729 (2001); Irikova, et al., Acta Physiologiae Plantarum, 33, 1559-1570 (2011); Ochoa-Alejo and Ireta-Moreno, Scientia Horticulturae, 42:1-2, 21-28 (1990); Gunay and Rao, Plant Science Letters, 11:3-4, 365-372 (1978). It is clear from the literature that these methods of obtaining plants are “conventional” in the sense that they are routinely successfully used. Thus, another aspect of this disclosure is to provide cells which upon growth and differentiation produce pepper plants having the physiological and morphological characteristics of hybrid pepper ‘SMK26’.

### Double Haploids

One way to obtain homozygous plants without the need for crossing and/or backcrossing is to produce haploids and then double the chromosomes to form doubled haploids. Haploid plants may be artificially induced via chemical treatments or by crossing plants with inducer lines (Seymour et al. 2012, PNAS vol. 109, pg. 4227-4232; Zhang et al., 2008 Plant Cell Rep. Dec 27 (12) 1851-60), and can also occur spontaneously. The production of haploid progeny can occur via a variety of mechanisms which can affect the distribution of chromosomes during gamete formation. A common technique to induce the formation of double haploid plants is with a chromosome doubling treatment such as colchicine (El-Hennawy et al., 2011 Vol 56, issue 2 pg. 63-72; Doubled Haploid Production in Crop Plants 2003 edited by Maluszynski ISBN 1-4020-1544-5). Other chromosome doubling agents that may be used include anti-mitotic agents including, but not limited to, trifluralin, oryzalin, amiprophos-methyl, and other polyploidy inducing agent(s). When creating a polyploid for breeding purposes, the layer of meristematic cells that give rise to the gametophytic tissue needs to be doubled. To optimize the probability of successful doubling, a high number of small, actively growing meristems are treated. Colchicine concentrations may vary depending on the tissue and species, but may be used, for example, at a concentration of 0.1% to 2.0%. Methods for treating seeds with colchicine or other spindle fiber inhibitors are well-known in the art, as discussed in Poehlman, J. M., Breeding Field Crops, University of Missouri, Holt, Rinehart and Winston Inc. (1966); Watts, L., Flower and Vegetable Plant Breeding, Grower Books (1980); Callaway D. J. and Callaway M. B., Breeding Ornamental Plants, Timber Press Inc. (2000). In addition, the chromosome complements of haploids sometimes double spontaneously to produce homozygous doubled haploids (DHs). Mixoploids, which are plants which contain cells having different ploidies, can sometimes arise, and may represent plants that are undergoing chromosome doubling so as to these plants may spontaneously produce doubled haploid tissues, organs, shoots, floral parts or plants. DHs are particularly valuable in breeding, as this technique allows the creation of true breeding lines without the need for multiple generations of selfing and selection. True breeding lines can be produced in as little as one generation using DHs.

Haploid embryos may be produced from microspores, pollen, anther cultures, or ovary cultures. The haploid embryos may then be doubled autonomously, or by chemical treatments (e.g., colchicine treatment). Alternatively, haploid embryos may be grown into haploid plants and treated to induce chromosome doubling. In either case, fertile homozygous plants are obtained. In accordance with the disclosure, any such techniques may be used to achieve a homozygous line derived from hybrid pepper ‘SMK26’.

Means for preparing pepper DH plants are well known in the art. By way of example, Kele, D. et al, “Effect of Pepper Types on Obtaining Spontaneous Doubled Haploid Plants via Anther culture” HortScience, pp1671-1676 (2015), Olszewska, D. et al, “The assessment of doubled haploid lines obtained in pepper (Capsicum annuum L.) anther culture” Folia Horticulturae, pp 93-99 (2011), and M. Maluszynski et al. (eds), Doubled Haploid Production in Crop Plants, (2003) describe certain techniques, which may be used. Ploidy changes have been shown to be easy to induce in Capsicum species, and an octoploid C. annuum has been reported (Panda et al, Theor. Appl. Genet. 68: 567-570 (1984). Thus, another aspect of this disclosure is to prepare DH plants from hybrid pepper ‘SMK26’. These DH plants may be used in breeding and/or in the production of polyploid plants of any ploidy level.

### Additional Breeding Methods

The hybrid pepper of the disclosure can also be used for transformation where exogenous genes are introduced and expressed by the cultivar of the disclosure. Genetic variants of ‘SMK26’ created either through traditional breeding methods or through transformation of hybrid pepper ‘SMK26’ by any of a number of protocols known to those of skill in the art are intended to be within the scope of this disclosure.

Mutations for use in mutation breeding can be induced in plants by using mutagenic chemicals such as ethyl methane sulfonate (EMS), by irradiation of plant material with gamma rays or fast neutrons, or by other means. The resulting nucleotide changes are random, but in a large collection of mutagenized plants the mutations in a gene of interest can be readily identified by using the TILLING (Targeting Induced Local Lesions IN Genomes) method (McCallum et al. (2000) Targeted screening for induced mutations. Nat. Biotechnol. 18, 455-457, and Henikoff et al. (2004) TILLING. Traditional mutagenesis meets functional genomics. Plant Physiol. 135, 630-636). The principle of this method is based on the PCR amplification of the gene of interest from genomic DNA of a large collection of mutagenized plants in the M2 generation. By DNA sequencing or by looking for point mutations using a single-strand specific nuclease, such as the CEL-I nuclease (Till et al. (2004) Mismatch cleavage by single-strand specific nucleases. Nucleic Acids Res. 32, 2632-2641), the individual plants that have a mutation in the gene of interest are identified. By screening many plants, a large collection of mutant alleles is obtained, each giving a different effect on gene expression or enzyme activity. The gene expression or protein levels can for example be tested by transcript analysis levels (e.g., by RT-PCR) or by quantification of protein levels with antibodies. Plants with the desired reduced gene expression or reduced protein expression are then back-crossed or crossed to other breeding lines to transfer only the desired new allele into the background of the crop wanted.

Genes of interest for use in breeding may also be edited using gene editing techniques including transcription activator-like effector nuclease (TALEN) gene editing techniques, Clustered Regularly Interspaced Short Palindromic Repeat (CRISPR/Cas9) gene editing techniques, and/or zinc-finger nuclease (ZFN) gene editing techniques. For this, transgenic plants are generated expressing one or more constructs targeting the gene of interest. These constructs may include, without limitation, an anti-sense construct, an optimized small-RNA construct, an inverted repeat construct, a targeting construct, a guide RNA construct, a construct encoding a targeting protein, and/or a combined sense-anti-sense construct, and may work in conjunction with a nuclease, an endonuclease, and/or an enzyme, so as to downregulate the expression of a gene of interest.

One of ordinary skill in the art of plant breeding would know how to evaluate the traits of two plant varieties to determine if there is no significant difference between the two traits expressed by those varieties. For example, see Fehr and Walt, Principles of Cultivar Development, p. 261-286 (1987). Thus the disclosure includes hybrid pepper ‘SMK26’ progeny pepper plants including a combination of at least two ‘SMK26’ traits selected from the combination of traits listed in the Overview of the Hybrid ‘SMK26’, so that said progeny pepper plant is not significantly different for said traits than pepper ‘SMK26’ as determined at the 5% significance level when grown in the same environmental conditions and/or may be characterized by percent similarity or identity to hybrid pepper ‘SMK26’ as determined by SSR markers. Using techniques described herein, molecular markers may be used to identify said progeny plant as a hybrid pepper ‘SMK26’ progeny plant. Mean trait values may be used to determine whether trait differences are significant, and preferably the traits are measured on plants grown under the same environmental conditions. Once such a variety is developed its value is substantial since it is important to advance the germplasm base as a whole in order to maintain or improve traits such as yield, disease resistance, pest resistance, and plant performance in extreme environmental conditions. As used herein, the term “plant” includes plant cells, plant protoplasts, plant cell tissue cultures from which pepper plants can be regenerated, plant calli, plant clumps and plant cells that are intact in plants or parts of plants, such as fruit, leaves, pollen, embryos, cotyledons, hypocotyl, roots, root tips, anthers, pistils, flowers, seeds, stems and the like.

The use of the terms “a,” “an,” and “the,” and similar referents in the context of describing the invention (especially in the context of the following claims) are to be construed to cover both the singular and the plural, unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. For example, if the range 10-15 is disclosed, then 11, 12, 13, and 14 are also disclosed. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

While a number of exemplary aspects and embodiments have been discussed above, those of skill in the art will recognize certain modifications, permutations, additions, and sub-combinations thereof. It is therefore intended that the following appended claims and claims hereafter introduced are interpreted to include all such modifications, permutations, additions, and sub-combinations as are within their true spirit and scope.

### Deposit Information

A deposit of at least 625 seeds of the hybrid pepper variety ‘SMK26’ was made with the National Collections of Industrial, Food and Marine Bacteria Ltd. (NCIMB Ltd.), Wellheads Place, Dyce, Aberdeen, AB21 7 GB, United Kingdom, and assigned NCIMB Number X1. The seeds deposited with the NCIMB on DATE were obtained from the seed of the variety maintained by BreedX Ltd., Kibbutz Givat Brenner 6094800, Israel since prior to the filing date of the application. Access to this deposit will be available during the pendency of this application to persons determined by the Commissioner of Patents and Trademarks to be entitled thereto under 37 C.F.R. § 1.14 and 35 U.S.C. § 122. Upon issuance, the Applicant will make the deposit available to the public consistent with all of the requirements of 37 C.F.R. § 1.801-1.809. This deposit of the hybrid pepper variety ‘SMK26’ will be maintained in the NCIMB, which is a public depository, for a period of 30 years, or at least 5 years after the most recent request for a sample of the deposit, or for the effective life of the patent, whichever is longer, and will be replaced if it becomes nonviable during that period. Applicant has no authority to waive any restrictions imposed by law on the transfer of biological material or its transportation in commerce. Applicant does not waive any infringement of rights granted under this patent or under the Plant Variety Protection Act (7 USC 2321 et seq.).

1. A seed of hybrid pepper designated as ‘SMK26’, representative sample of seed having been deposited under NCIMB Accession Number 44607.

2. A pepper plant produced by growing the seed of claim 1.

3. A plant part from the plant of claim 2, wherein said part is a leaf, an ovule, a pollen grain, a fruit, or a cell.

4. The plant part of claim 3, wherein said part is a fruit.

5. A pepper plant having all the physiological and morphological characteristics of the pepper plant of claim 2.

6. A plant part from the plant of claim 5, wherein said part is a leaf, an ovule, a pollen grain, a fruit, or a cell.

7. A pollen grain or an ovule of the plant of claim 2.

8. A protoplast produced from the plant of claim 2.

9. A tissue or cell culture produced from protoplasts or cells from the plant of claim 2, wherein said cells or protoplasts are produced from a plant part selected from the group consisting of root, root tip, meristematic cell, stem, hypocotyl, petiole, cotyledon, leaf, flower, anther, pollen, pistil, and fruit.

10. A pepper plant regenerated from the tissue or cell culture of claim 9, wherein the plant has all of the morphological and physiological characteristics of a pepper plant produced by growing hybrid pepper seed designated as ‘SMK26’, representative sample of seed having been deposited under NCIMB Accession Number 44607.

11. A method for producing a doubled haploid plant, comprising:
(a) producing a haploid genome from the seed of claim 1 or from a plant produced by growing the seed of claim 1;
(b) doubling the haploid genome to produce a doubled haploid genome; and
(c) producing a plant comprising the doubled haploid genome, thereby producing a doubled haploid plant.

12. A doubled haploid plant produced from the method of claim 11.

13. A plant part from the plant of claim 12, wherein said part is a leaf, an ovule, a pollen grain, a fruit, a cell, or a seed.

14. A method of producing a seed of ‘SMK26’-derived pepper seeds, comprising the steps:
a) producing a haploid genome from a hybrid pepper designated as ‘SMK26’, representative sample of seed having been deposited under NCIMB Accession Number 44607;
b) doubling the haploid genome to produce a doubled haploid genome;
c) producing a plant comprising the doubled haploid genome, thereby producing a doubled haploid ‘SMK26’ pepper;
d) crossing the doubled haploid ‘SMK26’ pepper with itself or a second pepper plant; and
e) whereby seed of a ‘SMK26’-derived pepper plant forms.

15. The method of claim 14, further comprising the steps of:
f) crossing a plant grown from ‘SMK26’-derived pepper seed with itself or a second pepper plant to yield additional ‘SMK26’-derived pepper seed;
g) growing the additional ‘SMK26’-derived pepper seed of step (c) to yield additional ‘SMK26’-derived pepper plants; and
h) repeating steps (f) and (g) for an additional 3-10 generations to generate further ‘SMK26’-derived pepper plants.

16. A pepper plant or seed produced from the method of claim 14, wherein the pepper plant or seed is a doubled haploid or a polyploid of any ploidy level.

17. A method of vegetatively propagating a plant produced by growing the seed of claim 1, the method comprising the steps of:
a) collecting tissue capable of being propagated from a plant of hybrid pepper ‘SMK26’, representative sample of seed having been deposited under NCIMB Accession Number 44607;
b) cultivating the tissue to obtain proliferated shoots; and
c) rooting the proliferated shoots to obtain rooted plantlets.

18. The method of claim 17, further comprising step (d) growing plants from the rooted plantlets.

19. A method of producing a pepper fruit, said method comprising growing the plant of claim 2 until it sets at least one fruit, and harvesting the fruit.

20. A method of vegetatively propagating a plant of hybrid pepper ‘SMK26’, the method comprising the steps of: (a) collecting tissue capable of being propagated from a plant of hybrid pepper ‘SMK26’, representative seed of said hybrid pepper ‘SMK26’ having been deposited under NCIMB Accession Number 44607; and (b) producing a rooted plant from said tissue.
